(19) United States
(12) Patent Application Publication
(10) Pub. No.: US 2006/0079927 A1
(43) Pub. Date: Apr. 13, 2006

(54) Detachable joint catheter and method

(75) Inventors: William F. Kaemmerer, Edina, MN (US); Kenneth T. Heruth, Edina, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., A Delaware Corporation, Santa Rosa, CA (US)

(21) Appl. No.: 10/961,964

(22) Filed: Oct. 8, 2004

(51) Int. Cl. A61M 29/00

(52) U.S. Cl. 606/200

(57) ABSTRACT

A tip delivery system includes a tip, a tip support structure and a dissolvable joint connecting the tip to the tip support structure. To separate the tip from the tip support structure, a dissolving liquid is delivered to the dissolvable joint. The dissolving liquid reacts, e.g., enzymatically, with the dissolvable joint and dissolves the dissolvable joint.

### BACKGROUND OF THE INVENTION

1. Field of the Invention

The present invention relates generally to medical devices and methods. More particularly, the present invention relates to a method and device for the placement of a structure in a human body.

2. Description of Related Art

Vascular aneurysms are the result of abnormal dilation of a blood vessel, usually resulting from disease and/or genetic predisposition, which weakens the arterial wall and allows it to expand. The weakened areas of the arterial wall caused by an aneurysm are problematic because they are subject to rupture and if a rupture occurs, the aneurysm can prove fatal.

Several procedures and methods have been established for the exclusion of aneurysms. One such method involved the insertion of a coil within the aneurysm itself. Using coil insertion methods, the coil precipitated the formation of a thrombus, or clot, within the aneurysm. The thrombus then partially, or completely, occluded the aneurysm. In this manner, blood from the parent artery (or vessel) was prevented from flowing into, and circulating within, the aneurysm. Consequently, pressure on the weakened arterial wall at the aneurysm site was reduced, as was the risk of rupture.

To deliver the coil to the aneurysm, the coil was located at the distal end of a delivery catheter. The coil was positioned within the aneurysm. The coil was then detached from the distal end of the delivery catheter. The delivery catheter was then withdrawn leaving the coil within the aneurysm.

The coil was attached to the distal end of the delivery catheter with solder. To detach the coil from the distal end of the delivery catheter, an electrical current was applied to the solder. Application of the electrical current heated the solder, which upon reaching a sufficiently high temperature, melted. Melting of the solder detached the coil from the distal end of the delivery catheter.

While at times an effective treatment, the coil insertion method caused the body tissue surrounding the solder to become heated and sometimes burn. Further, the molten solder sometimes became separated from both the coil and the distal end of the delivery catheter and was released into the vasculature of the patient. In some anatomical placements, heating of the surrounding body tissue and the risk of debris from the molten solder is contraindicated.

### SUMMARY OF THE INVENTION

A tip delivery system includes a tip, a tip support structure and a dissolvable joint connecting the tip to the tip support structure. To separate the tip from the tip support structure, a dissolving liquid is delivered to the dissolvable joint. The dissolving liquid reacts, e.g., enzymatically, with the dissolvable joint and dissolves the dissolvable joint.

The dissolvable joint is dissolved by the dissolving liquid and without heating the dissolvable joint above body temperature. Accordingly, there is no risk of heating or burning the surrounding body tissue.

Further, the dissolvable joint is completely dissolved by the dissolving liquid. Accordingly, there is essentially no risk of debris from the dissolvable joint.

In another embodiment, the dissolvable joint and the dissolving liquid are biocompatible, e.g., are not contraindicated for the patient. Accordingly, even if debris is formed from the dissolvable joint, there is no contraindication for the patient.

### BRIEF DESCRIPTION OF THE DRAWINGS

FIG. 1 is a cross-sectional view of a distal end of a tip delivery system in accordance with one embodiment of the present invention;

FIGS. 2 and 3 are cross-sectional and perspective views of the distal end of the tip delivery system of FIG. 1 at a later stage during deployment of a tip;

FIG. 4 is a cross-sectional view of the distal end of the tip delivery system of FIG. 2 at a later stage of deployment of the tip;

FIGS. 5 and 6 are cross-sectional and perspective views of a distal end of a tip delivery system in accordance with another embodiment of the present invention; and

FIG. 7 is a cross-sectional view of the distal end of the tip delivery system of FIG. 6 at a later stage of deployment of a tip.

Common reference numerals are used throughout the drawings and detailed description to indicate like elements.

### DETAILED DESCRIPTION

Referring to FIG. 2, a tip delivery system 100 includes a tip 106, a tip support structure 104 and a dissolvable joint 108 connecting tip 106 to tip support structure 104. To separate tip 106 from tip support structure 104, a dissolving liquid 206 is delivered to dissolvable joint 108. Dissolving liquid 206 reacts, e.g., enzymatically, with dissolvable joint 108 and dissolves dissolvable joint 108 separating tip 106 from tip support structure 104 as shown in FIG. 4.

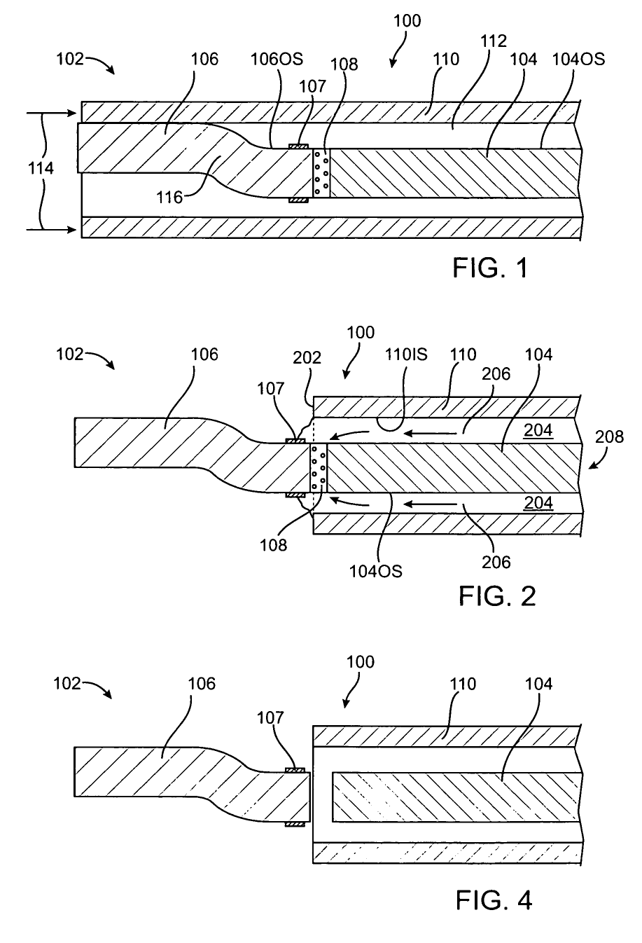

More particularly, FIG. 1 is a cross-sectional view of a distal end 102 of a tip delivery system 100, sometimes called a detachable joint catheter, in accordance with one embodiment of the present invention. As used herein, the proximal end of the delivery system is referenced with respect to (closest to) the operator's handle while the distal end of the delivery system is referenced with respect to the opposite of (furthest away from) the operator's handle.

Referring now to FIG. 1, tip delivery system 100 includes a wire 104 coupled to a tip 106 by a dissolvable joint 108, sometimes called a means for connecting tip 106 to wire 104. Tip delivery system 100 further includes a sheath 110 comprising a lumen 112. In this example, wire 104, tip 106, and dissolvable joint 108 are located within lumen 112 of sheath 110. In FIG. 1, sheath 110 is shown prior to retraction, i.e., prior to movement in the proximal direction 114.

In one example, wire 104 is a solid but flexible cylindrical rod and tip 106 is a coil. As shown in FIG. 1, tip 106 has a bend 116 facilitating anchoring of tip 106 within an aneurysm. However, tip 106 can be formed in any one of a number of shapes, e.g., circular, wound, oval, spiral, or other shape.

Further, tip 106 can be formed of a shaped memory material. In accordance with this example, tip 106 is restrained within sheath 110 and upon retraction of sheath 110 and exposure of tip 106, tip 106 assumes its shape.

Still further, tip 106 can be formed of a polymer. In accordance with this example, a band 107, sometimes called a ring, of metal, metal powder slurry, e.g., barium sulfate in room temperature vulcanizing silicon (RTV), is formed on tip 106 to make tip 106 radiopaque, e.g., visible with X-ray, fluoroscopy, MRI or other imaging technique. Band 107 can also be formed of magnetic material as another example. Still further, an RF coil can be placed on tip 106, for example, for use with the stealth station image-guided surgery technology developed by Medtronic Surgical Navigation Technologies, a division of Medtronic, Inc.

Dissolvable joint 108 is dissolvable. More particularly, upon contact of dissolvable joint 108 with a dissolving liquid applied through lumen 112, sometimes called a means for delivering a dissolving liquid, of sheath 110, dissolvable joint 108 dissolves.

Dissolvable joint 108 connects tip 106 to wire 104. In one example, dissolve joint 108 is cylindrical or disk shaped. In accordance with this example, dissolvable joint 108 is an extension of wire 104, e.g., has an outer diameter equal to an outer diameter of wire 104. Accordingly, dissolve joint 108 is between tip 106 and wire 104, which are spaced apart from one another by dissolvable joint 108. Wire 104 is sometimes called a tip support structure.

In another example, dissolvable joint 108 is a tape, sometimes called a film or sheet. In accordance with this example, dissolvable joint 108 is wrapped around outer surfaces 106OS, 104OS of tip 106 and wire 104, respectively. Thus, tip 106 is in abutting contact with wire 104, but can also be spaced apart from wire 104.

In one example, dissolvable joint 108 is solid, sometimes called a non-perforated and non-porous solid. However, in other examples, dissolvable joint 108 is semi-solid. As a semi-solid, dissolvable joint 108 is: (1) formed of loose fibers, e.g., similar in structure to cotton candy; (2) perforated; or (3) formed with pores, e.g., similar in structure to a sponge. By forming dissolvable joint 108 as a semi-solid, in one example, the dissolvability of dissolvable joint 108 is maximized because the surface area of dissolvable joint 108 for contact with the dissolving liquid is maximized.

In one specific example, dissolvable joint 108 is a polymer and the dissolving liquid is an enzyme, which dissolves the polymer through enzymatic reaction. An enzymatic reaction, sometimes called an enzyme reaction, is a chemical reaction involving the synthesis or degradation of a substrate molecule in which a protein molecule, i.e., the enzyme, serves as a catalyst for the reaction, enabling the reaction to occur at a fast rate at a physiologic temperature. The enzyme itself is not consumed or destroyed by the enzymatic reaction.

For example, dissolvable joint 108 is hyaluronic acid, e.g., cross-linked, and the dissolving liquid is a physiologic buffer solution containing an effective amount of the enzyme hyaluronidase in solution. Some synonyms for hyaluronidase are as follows: hyaluronoglucosaminidase, hyaluronoglucosidase, chondroitinase, and chondroitinase I. The protein number for all of these is: EC 3.2.1.35.

Hyaluronidase is not toxic, as it is a substance that is present in the body normally, including in human serum and saliva. There are several human genes for various hyaluronidases, of which HYAL1 is the gene for the form of hyaluronidase found in human serum. Hyaluronidase is used medically as a co-factor to enhance anesthesia administered to the eye for cataract surgery, and as a treatment to prevent edema following tissue transplantation.

A commercially available form of hyaluronidase is FDA approved and marketed under the trade name Vitrase, for use in enhancing the dispersion of injected drugs, e.g., subcutaneous injections.

Note that a safety concern is that some people can have pre-existing allergies to hyaluronidase, or can be sensitized to hyaluronidase and thus develop a reaction with repeated dosing. This possibility can be ruled out with a “skin prick” test done several days prior to the use of tip delivery system 100, to verify that the use of hyaluronidase is not contraindicated for the patient due to allergy.

Further, Hyaluronidase is rapidly inactivated in the human bloodstream. Human blood contains several circulating inhibitors of hyaluronidase, some of which have been identified (genes are known). Heparin is also known to be an inactivator of hyaluronidase.

In one example, the rate of dissolution of joint 108 depends upon how the hyaluronic acid is formulated (e.g., how much it is esterified) as those of skill in the art will understand in light of this disclosure. Illustratively, the hyaluronic acid of joint 108 is esterified such that joint 108 dissolves in a matter of minutes. Those of skill in the art will understand that the trade-off is between durability of joint 108 (for handling and insertion) versus how rapidly joint 108 will be enzymatically dissolved.

In one example, to enhance the action of the dissolving liquid and speed the dissolving action, the dissolving liquid is an acidic solution, e.g., the pH of the dissolving liquid is set to an acidic level, such as 3.5 to 4.0. The buffer solution in which the hyaluronidase is administered can be formulated to provide this more acidic pH. In accordance with this example, the buffer solution is sometimes called an acidic buffer.

However, the buffer solution in which the hyaluronidase is administered can also formulated to provide a neutral pH, e.g., 7.0. In accordance with this example, the buffer solution is sometimes called a physiologic buffer.

Illustratively, distal end 102 of tip delivery system 100 including tip 106 are maneuvered into an aneurysm for example through an artery, e.g., the carotid artery. Sheath 110 is retracted, i.e., moved in proximal direction 114, uncovering and exposing tip 106.

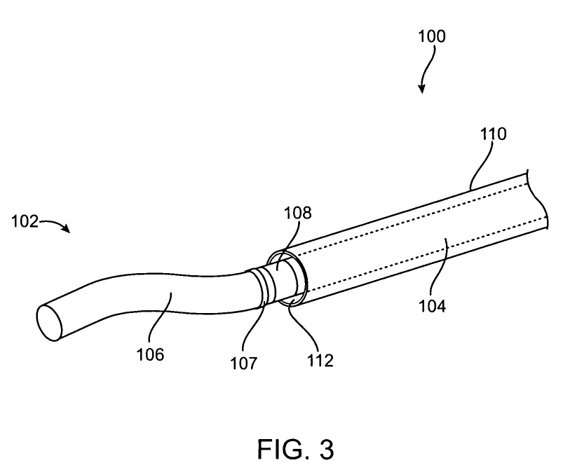

FIGS. 2 and 3 are cross-sectional and perspective views of distal end 102 of tip delivery system 100 of FIG. 1 at a later stage during deployment of tip 106. Referring now to FIGS. 2 and 3 together, sheath 110 has been retracted to expose tip 106. More particularly, sheath 110 has been retracted such that a distal end 202 of sheath 110 is adjacent dissolvable joint 108.

An inner cylindrical surface 1101S of sheath 110 and outer surface 1040S of wire 104 define an annular space 204. A dissolving liquid 206 is injected into annular space 204 at a proximal end 208 of tip delivery system 100. In light of this disclosure, those of skill in the art will understand that injection of dissolving liquid 206 into annular space 204 can be performed using any one of a number of well-known liquid injection techniques such as injecting dissolving liquid 206 through a port of a handle of delivery system 100.

Upon injection into annular space 204, dissolving liquid 206 flows distally through annular space 204 and contacts dissolvable joint 108. Dissolving liquid 206 reacts, enzymatically, with dissolvable joint 108 and dissolves dissolvable joint 108. In one example, at body temperature, dissolving liquid 206 dissolves dissolvable joint 108 in a short time, e.g., in minutes or otherwise in an amount of time acceptable for the particular procedure being performed.

Dissolvable joint 108 is dissolved by dissolving liquid 206 and without heating dissolvable joint 108 above body temperature. Accordingly, there is no risk of heating or burning the surrounding body tissue.

Further, dissolvable joint 108 is completely dissolved by dissolving liquid 206. Accordingly, there is essentially no risk of debris from dissolvable joint 108.

In another embodiment, dissolvable joint 108 and dissolving liquid 206 are biocompatible, e.g., are not contraindicated for the patient. Accordingly, even if debris is formed from dissolvable joint 108, there is no counter-indication for the patient.

FIG. 4 is a cross-sectional view of distal end 102 of tip delivery system 100 of FIG. 2 at a later stage of deployment of tip 106. Referring now to FIGS. 2 and 4 together, dissolving liquid 206 has dissolved dissolvable joint 108. Accordingly, tip 106 is detached from wire 104. Once detached, tip delivery system 100 including wire 104 and sheath 110 is withdrawn from the patient leaving tip 106 where deployed.

In one example, tip delivery system 100 is a neural or neurovascular catheter and tip 106 is a coil deployed within an aneurysm, e.g., a neurovascular aneurysm. Tip 106 precipitates the formation of a thrombus, or clot, within the aneurysm. The thrombus partially, or completely, occludes the aneurysm. In this manner, blood from the parent artery (or vessel) is prevented from flowing into, and circulating within, the aneurysm. Consequently, pressure on the weakened arterial wall at the aneurysm site is reduced, as is the risk of rupture.

In another embodiment, tip 106 comprises a biocompatible tissue scaffold, e.g., a cell growth medium. Tip 106 further includes cells such as neurons or neuronal precursors in or on the tissue scaffold.

In accordance with this example, tip 106 is positioned and deployed within the brain, e.g., through the carotid artery. Once deployed, the cells, sometimes called the bioactive substance, of tip 106 diffuse to the surrounding brain tissue. In this manner, cell survival is maximized and the cells are delivered over an extended period of time.

In one particular example, neural cells are transplanted into the brain using tip 106 for the treatment of Parkinson's disease, Huntington's disease, stroke, or other indications for neuronal replacement and repair.

In other examples, tip 106 comprises a drug. For example, tip 106 releases a drug(s) over a period of time. Illustratively, tip 106 dissolves releasing a drug. In another example, tip 106 includes a drug suspension matrix from which a drug diffuses out over time. Tip 106 is delivered to regions of the brain, e.g., through the carotid artery.

In one specific example, tip 106 is delivered to the subtantia nigra or the subthalamic nucleus. Tip 106 includes dopamine, which is delivered to these regions for treatment of Parkinson's disease.

In another specific example, tip 106 is delivered to the locus of seizures. Tip 106 includes an anti-convulsant such as phenytoin or carbamazepine, which is delivered to the locus for the treatment of epilepsy.

In yet another specific example, tip 106 is delivered to the site of an inoperable brain tumor. Tip 106 includes chemotherapy agents such as Bleomycin, Flouracil (5-FU), Floxuridine (FUDR), doxorubicin, which is delivered to site for treatment of the tumor.

In yet another specific example, tip 106 is delivered to a cerebral pain relief site(s) such as the medulla, the periaquaductal gray, the thalamus, and/or the motor cortex. Tip 106 includes opiods, which are delivered to the pain relief site(s) for treatment of pain.

In yet another example, tip 106 is delivered to a subcutaneous or abdominal site in a patient with diabetes. Tip 106 includes a tissue scaffold containing insulin-producing cells, such as pancreatic islets or beta cells.

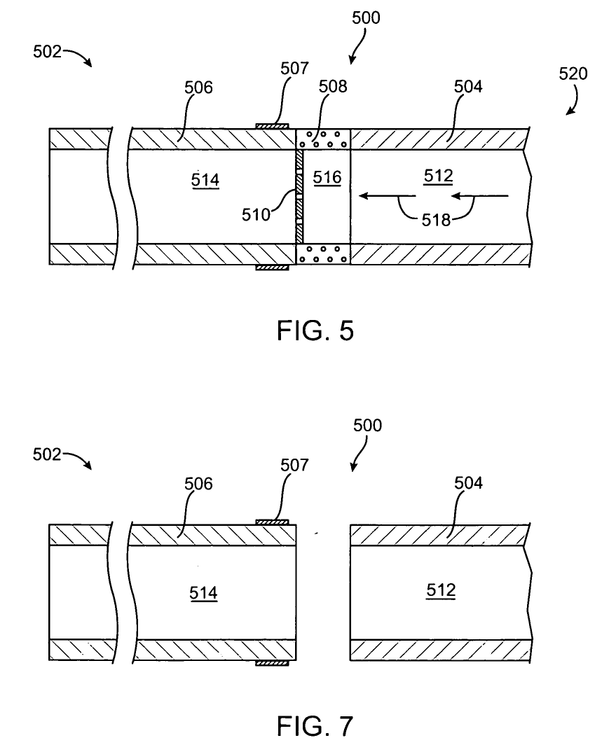

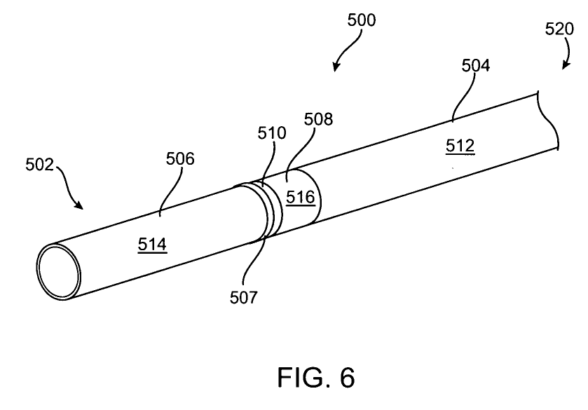

FIGS. 5 and 6 are cross-sectional and perspective views of a distal end 502 of a tip delivery system 500 in accordance with another embodiment of the present invention.

Referring now to FIGS. 5 and 6 together, tip delivery system 500 includes a tube 504 coupled to a tip 506 by a dissolvable joint 508. Tip delivery system 500 further includes a dissolvable membrane 510.

As shown, tube 504, tip 506, and dissolvable joint 508 include lumens 512, 514, 516, respectively. Dissolvable membrane 510 extends across lumen 516 of dissolvable joint 508 and restricts the flow of dissolving liquid at dissolvable joint 508. For example, dissolvable membrane 510 is perforated as illustrated in FIG. 5 allowing some fluid flow across dissolvable membrane 510.

Dissolvable joint 508 is dissolvable. More particularly, upon contact of dissolvable joint 508 with a dissolving liquid applied through lumen 512, sometimes called a means for delivering a dissolving liquid, of tube 504, dissolvable joint 508 dissolves.

Dissolvable joint 508 connects tip 506 to tube 504. Thus, tube 504 is sometimes called a tip support structure. In one example, dissolvable joint 508 of FIG. 5 is similar to dissolvable joint 108 of FIG. 1 except dissolvable joint 508 includes lumen 516. For example, dissolvable joint 508 is hyaluronic acid and the dissolving liquid is a physiologic buffer solution containing an effective amount of the enzyme hyaluronidase in solution.

In one example, tip 506 is a polymeric tube used to maintain the patency of an opening. In one specific example, tip 506 is a polymeric tube used as an endoventricular shunt across an obstruction for the treatment of hydrocephalus. More particularly, tip 506 maintains the patency of an opening between the 3rd ventricle and the interpeduncular cistern, thus relieving pressure from the 3rd ventricle by allowing cerebrospinal fluid to flow across the obstruction.

In accordance with this example, a band 507, sometimes called a ring, of metal, metal powder slurry, e.g., barium sulfate in room temperature vulcanizing silicon (RTV), is formed on tip 506 to make tip 506 radiopaque, e.g., visible with X-ray, fluoroscopy, MRI or other imaging technique. Band 507 can also be formed of magnetic material as another example. Still further, an RF coil can be placed on tip 506, for example, for use with the stealth station image-guided surgery technology developed by Medtronic Surgical Navigation Technologies, a division of Medtronic, Inc.

Illustratively, distal end 502 of tip delivery system 500 including tip 506 are maneuvered into an opening between the 3rd ventricle and the interpeduncular cistern, e.g., through the subarachnoid space or a burr hole in the skull.

Referring now to FIG. 5, a dissolving liquid 518 is injected into lumen 512 of tube 504 at a proximal end 520 of tip delivery system 500. In light of this disclosure, those of skill in the art will understand that injection of dissolving liquid 518 into lumen 512 of tube 504 can be performed using any one of a number of well-known liquid injection techniques such as injecting dissolving liquid 518 through a port of a handle of delivery system 500.

Upon injection into lumen 512 of tube 504, dissolving liquid 518 flows distally through lumen 512 of tube 504 and contacts dissolvable joint 508. Dissolving liquid 518 reacts, enzymatically, with dissolvable joint 508 and dissolves dissolvable joint 508. In one example, at body temperature, dissolving liquid 518 dissolves dissolvable joint 508 in a short time, e.g., in minutes or otherwise in an amount of time acceptable for the particular procedure being performed. Dissolving liquid 518 is similar to or the same as dissolving liquid 206 of FIG. 2.

Dissolvable joint 508 is dissolved by dissolving liquid 518 and without heating dissolvable joint 508 above body temperature. Accordingly, there is no risk of heating or burning the surrounding body tissue.

Further, dissolvable joint 508 is completely dissolved by dissolving liquid 518. Accordingly, there is essentially no risk of debris from dissolvable joint 508.

In another embodiment, dissolvable joint 508 and dissolving liquid 518 are biocompatible, e.g., are not contraindicated for the patient. Accordingly, even if debris is formed from dissolvable joint 508, there is no counter-indication for the patient.

Dissolvable membrane 510 enhances the application of dissolving liquid 518 to dissolvable joint 508. Specifically, dissolvable membrane 510 restricts the flow of dissolving liquid 518 at dissolvable joint 508. Accordingly, dissolving membrane 510 minimizes the amount of dissolving liquid 518 necessary to dissolve dissolvable joint 508. However, dissolving membrane 510 is optional, and in one example, is not formed.

In accordance with one example, dissolving membrane 510 is formed of a same material as dissolving joint 508. For example, dissolving membrane 510 and dissolving joint 508 are integral, i.e., are parts of the same piece and not a plurality of separate pieces connected together. However, in another example, dissolving membrane 510 and dissolving joint 508 are separate pieces connected together.

By forming dissolving membrane 510 of the same material as dissolving joint 508, e.g., hyaluronic acid, dissolving membrane 510 dissolves simultaneously with dissolving joint 508 upon contact with dissolving liquid 518.

FIG. 7 is a cross-sectional view of distal end 502 of tip delivery system 500 of FIG. 5 at a later stage of deployment of tip 506. Referring now to FIGS. 5 and 7 together, dissolving liquid 518 has dissolved dissolvable joint 508 and dissolvable membrane 510. Accordingly, tip 506 is detached from tube 504. Once detached, tip delivery system 500 including tube 504 is withdrawn from the patient leaving tip 506 where deployed, e.g., in an opening. Once deployed, fluid, e.g., cerebrospinal fluid, flows through lumen 514 of tip 506.

This disclosure provides exemplary embodiments according to the present invention. Numerous variations, whether explicitly provided for by the specification or implied by the specification or not, such as variations in structure, dimension, type of material and manufacturing process may be implemented by one of skill in the art in view of this disclosure.

What is claimed is:

1. A structure comprising:
a tip;
a tip support structure; and
a dissolvable joint coupling said tip to said tip support structure, wherein application of a dissolving liquid to said dissolvable joint dissolves said dissolvable joint through enzymatic reaction.

2. The structure of claim 1 further comprising a sheath comprising a lumen, said tip support structure and said dissolvable joint being located within said lumen.

3. The structure of claim 1 wherein said tip comprises a bend.

4. The structure of claim 1 wherein said tip comprises a shape selected from the group consisting of a circular, wound, oval, and spiral shape.

5. The structure of claim 1 wherein said tip comprises a shaped memory material.

6. The structure of claim 1 wherein said dissolvable joint is cylindrical shaped.

7. The structure of claim 6 wherein said dissolvable joint is an extension of said tip support structure.

8. The structure of claim 1 wherein said tip and said tip support structure are spaced apart from one another by said dissolvable joint.

9. The structure of claim 1 wherein said tip support structure is a wire.

10. The structure of claim 1 wherein said dissolvable joint comprises a tape.

11. The structure of claim 10 wherein said dissolvable joint is wrapped around outer surfaces of said tip and said tip support structure.

12. The structure of claim 10 wherein said tip is in abutting contact with said tip support structure.

13. The structure of claim 1 wherein said dissolvable joint comprises a non-perforated and non-porous solid.

14. The structure of claim 1 wherein said dissolvable joint comprises a semi-solid.

15. The structure of claim 1 wherein said dissolve joint is formed of loose fibers.

16. The structure of claim 1 wherein said dissolve joint is perforated.

17. The structure of claim 1 wherein said dissolve joint is formed with pores.

18. The structure of claim 1 wherein said dissolvable joint comprises a polymer and said dissolving liquid comprises an enzyme, said enzyme dissolving said polymer.

19. The structure of claim 18 wherein said polymer comprises hyaluronic acid and said enzyme comprises hyaluronidase.

20. The structure of claim 1 wherein said tip comprises a coil.

21. The structure of claim 1 wherein said tip comprises a biocompatible tissue scaffold and a bioactive substance.

22. The structure of claim 21 wherein said bioactive substance comprises neural cells.

23. The structure of claim 21 wherein said bioactive substance comprises insulin-producing cells.

24. The structure of claim 1 wherein said tip comprises a drug.

25. The structure of claim 24 wherein said drug comprises dopamine.

26. The structure of claim 24 wherein said drug comprises an anti-convulsant.

27. The structure of claim 24 wherein said drug comprises a chemotherapy agent.

28. The structure of claim 24 wherein said drug comprises opiods.

29. The structure of claim 1 wherein said tip support structure comprises a tube comprising a lumen.

30. The structure of claim 29 wherein said dissolving liquid is delivered through said lumen of said tube.

31. The structure of claim 1 wherein said dissolvable joint comprises a lumen, said structure further comprising a dissolvable membrane extending across said lumen.

32. The structure of claim 1 wherein said tip comprises a tube.

33. The structure of claim 32 wherein said tube comprises a polymeric tube for maintaining a patency of an opening.

34. A method comprising:
delivering a dissolving liquid to a dissolvable joint coupling a tip to a tip support structure, wherein said dissolving liquid dissolves said dissolvable joint through enzymatic reaction separating said tip from said tip support structure.

35. The method of claim 34 further comprising positioning said tip within an aneurysm.

36. The method of claim 35 wherein said tip comprises a coil.

37. The method of claim 34 further comprising positioning said tip in an opening.

38. The method of claim 37 wherein said tip comprises a tube.

39. The method of claim 34 wherein said delivering a dissolving liquid to a dissolvable joint comprises injecting said dissolving liquid into an annular space defined by an outer surface of said tip support structure and an inner surface of a sheath.

40. The method of claim 34 wherein said delivering a dissolving liquid to a dissolvable joint comprises injecting said dissolving liquid into a lumen of said tip support structure.

41. The method of claim 40 further comprising restricting a flow of said dissolving liquid at said dissolvable joint.

42. The method of claim 41 wherein said flow of said dissolving liquid is restricted at said dissolve joint by a dissolvable membrane.

43. The method of claim 42 wherein said dissolving liquid dissolves said dissolvable membrane.

44. A structure comprising:
a means for coupling a tip to a tip support structure;
a means for delivering a dissolving liquid to said means for coupling, wherein said dissolving liquid dissolves said means for coupling through enzymatic reaction separating said tip from said tip support structure.
